(19) United States
(12) Patent Application Publication
(10) Pub. No.: US 2017/0119657 A1
(43) Pub. Date: May 4, 2017

(54) ANHYDROUS CONSUMABLE DEVICE FOR THE EXTEMPORANEOUS PREPARATION OF A SINGLE-DOSE COSMETIC COMPOSITION

(71) Applicant: GATTEFOSSE HOLDING, Saint Priest (FR)

(72) Inventor: Frèdèric DEMARNE, Marseille (FR)

(73) Assignee: GATTEFOSSE HOLDING, Saint Priest (FR)

(21) Appl. No.: 15/125,402

(22) PCT Filed: Mar. 5, 2015
(86) PCT No.: PCT/FR2015/050549

§ 371 Date: Sep. 12, 2016

(30) Foreign Application Priority Data
Mar. 21, 2014 (FR) 1452377

(51) Int. Cl. A61K 8/92; A61K 8/55; A61K 8/58; A61K 8/97; A61Q 19/00; A61K 8/37; A61K 8/34; A61K 8/11; A61K 8/73; A61K 8/65; A61K 8/06; A61K 8/33

(52) U.S. Cl. CPC A61K 8/92; A61K 8/06; A61K 8/922; A61K 8/553; A61K 8/585; A61K 8/97; A61K 8/33; A61K 8/375; A61K 8/342; A61K 8/37; A61K 8/11; A61K 8/73; A61K 8/731; A61K 8/65; A61Q 19/00; A61Q 19/007; A61K 2800/81; A61K 2800/592; A61K 2800/52; A61K 2800/30

(57) ABSTRACT

Anhydrous device in the form of a closed shell containing a fatty phase, the shell and the fatty phase constituting the necessary ingredients and in an amount adequate for the extemporaneous preparation of a single-use cosmetic emulsion, solely by adding water.

The present invention relates to a consumable anhydrous device comprising a fatty phase for the extemporaneous preparation, solely by adding water, of a single-dose cosmetic emulsion. It also relates to a method for the extemporaneous preparation of a single-dose cosmetic emulsion implementing the device of the invention. This method is intended to be implemented directly by the consumer of the cosmetic product immediately prior to its use.

Conventional cosmetic products are manufactured on an industrial scale in factories, packaged in containers of varying sizes and then sent to points of sale where they are sold to the final consumer after a more or less extended storage period. Containers used for cosmetic products (tubes, jars, bottles or others) generally have a capacity which corresponds to several weeks of cosmetic product use. Once purchased, the consumer stores the cosmetic product at home for a period of time often exceeding several months. This kind of storage takes place at room temperature without any special precautions, the cosmetic product can thus be exposed to high temperatures during summer or if stored in proximity to a heater.

However, cosmetic products, which are mostly oil-in-water emulsions have a limited lifespan. Indeed, the two phases of these emulsions have a tendency to separate after a certain period of time. This aging phenomenon is accelerated when the product is exposed to heat. In addition, the main active ingredients used for producing these cosmetics are often unstable and decompose when they are in prolonged contact with the aqueous phase of the emulsion.

To extend the life of cosmetic products by conserving the quality of the emulsion and its effectiveness, cosmetic products usually contain preservatives in order to stabilize them but also to prevent the growth of microorganisms. These preservatives, which are often synthetic chemicals such as parabens or other chemicals, are not well regarded by current users. They are considered toxic, responsible for allergies, and they are suspected of giving rise to harmful effects on health.

According to the current trend, a large number of consumers are looking for cosmetic products made with natural ingredients, with the fewest possible chemical excipients and preservatives. However, the removal of preservatives is inconsistent with the manner cosmetic products are currently manufactured, distributed, stored, and used by consumers.

To satisfy these consumers, cosmetic products made by the user at home have been proposed, following a recipe from ingredients purchase separately. However, these preparations are rather complex to implement. They require time and sometimes expensive equipment. Further, a large number of raw materials in relatively large amounts must be bought and then stored. Due to the financial investment required and the complexity of the proposed preparations which often discourage initially interested consumers, this trend has remained marginal so far. Furthermore, it does not solve the problem of the limited lifespan of those manufactured cosmetic products when they contain no preservatives.

In order to facilitate the manufacture of cosmetic products by consumers simplified manufacturing processes were proposed in prior art.

Thus, for example, EP 2038189 B1 is known which describes a process for the production of a cosmetic product according to which consumers manufacture this cosmetic product themselves, in amounts matching their household usage. To do so they use a machine within which they place one or several packaging units containing all the cosmetic ingredients needed for the manufacture of the product in previously measured quantities. The machine then automatically performs the mixing of all the cosmetic ingredients contained in the packaging units, with water, without the consumer having to measure quantities or weigh ingredients. This method simplifies the manufacture of a cosmetic product by the consumer since there is no longer need to weigh or measure the amounts of ingredients they are provided with. It is the machine that automatically mixes these ingredients with water. This method does however requires consumers to purchase a specific machine, essential to carry out the method described, the cost of which is significant. In addition, this machine is cumbersome, consumes energy to operate, and requires maintenance. Further, the intended packaging units, which comprise several separate tight compartments to separate the different components, are complex to produce resulting in significant cost. Thus, the purchase price of ingredients in these packaging units is high for consumers, and comparatively much more expensive than the cost of commercially available conventional cosmetic products.

Moreover, consumers interested in producing their cosmetic products at home are for the most part people looking for natural products for their cosmetics, and wanting to control and limit the use of chemical ingredients and preservatives. If their main motivation is the preservation of their health then it is usually associated with an ecological concern of preserving the environment. However, after use, the empty packaging units form much bulky and generally non-recyclable waste that poses an environmental problem and is ecologically offensive to these potential consumers who are usually proponents of the fight against excess packaging.

Finally, this prior patent intends to prepare cosmetic products in amounts meant for domestic use, i.e. in amounts comparable to those of packaging in which the product is usually sold to the end consumer in conventional stores. Once prepared, consumers will have to keep it for several weeks, which may prove problematic in the absence of preservatives.

The method and the kit described in US patent application 2004/0202684 are also known. According to this method, to obtain a cosmetic product, consumers must mix, in a receptacle provided in the kit, the contents of two or three containers, also provided in the kit, themselves. The first of these containers contains a mixture of anhydrous cosmetic active ingredients, the second an aqueous liquid, and the third a thickener and/or gelling agent that the consumer may add in varying amounts to obtain a cosmetic product with the final consistency they desire. According to alternative embodiments, the thickening agent may also be mixed with the anhydrous active ingredients of the first container. In this case, the kit comprises no more than two containers.

The purpose of this process is to be able to customize the cosmetic product manufactured by adding to it other ingredients derived from drinks or food (fruit or fresh vegetable juices, tea, coffee, milk, wine . . . ) whose components are particularly unstable.

This method may advantageously be carried out manually without use of a special machine, which makes it more affordable to consumers. However, the method is more complicated for the user who needs to add ingredients not included in the kit, for which they need to determine and measure themselves amounts to be added. Similarly, the ingredients provided in the containers of the kit do not necessarily have to be used entirely and therefore must be measured before addition, and then stored.

Furthermore, the problem of waste generated by empty containers after use of ingredients is not resolved. The amount of packaging to throw away is yet again increased by the fact that, according to this method, even the water needed for the preparation of the cosmetic product is packaged in a container provided in the kit which must be discarded after use.

Document CH 663725 A5 describes a process of instantly manufacturing ointment through mixing separate dose pouches containing constituent ingredients of an oily phase or an aqueous phase. The pouches are discharged into a tube-type container in which the mixing is carried out. The operator must select the different pouches and mix their content in order to obtain the final composition from a predetermined recipe.

Document GB 2 118 961 A describes a bath preparation in the form of pouches containing essentially surfactants. These pouches are made of PVA and are intended to dissolve in the bath water.

Document U.S. Pat. No. 5,326,564 describes dual-compartment capsules, with one of the compartments fillable with an anhydrous composition. The shell is reusable.

Document U.S. Pat. No. 4,292,304 A describes an oil-based anhydrous toothpaste, stored in gelatin capsules intended to be ingested. The capsule only serves as toothpaste packaging.

Document EP 453 683 A1 describes a cosmetic composition in the form of a gelatin capsule containing a cosmetic composition. The capsule is only used as a container.

Document US 2004/0234590 A1 describes capsules intended to be ingested. The shell is intended to dissolve in contact with an aqueous phase.

The object of the invention is to provide an alternative method of preparing a cosmetic product achievable by the user, which does not present the drawbacks of the aforementioned prior methods.

The method according to the invention is extremely easy to implement, with a simple operation of mixing, and without needing to use a machine. It requires no complicated ingredient preparation or respective amount measuring operation by the user.

This fast and inexpensive method is further ecological as it does not generate any waste.

The method according to the invention allows the user to extemporaneously prepare only the dose of the necessary cosmetic product, just before its application. The cosmetic product thus produced is not intended to be preserved and is intended to be used immediately after manufacture.

Thus, its efficiency is maximal and the use of preservatives or even stabilizers is not absolutely necessary. It is thus possible to very easily manufacture cosmetic products without preservatives, perfectly adapted to the expectations of consumers seeking cosmetic products made of “pure” active ingredients, i.e. without unnecessary additives.

More specifically, the invention proposes a consumable anhydrous device comprising a fatty phase, the device being intended for extemporaneous preparation, solely by adding water, of a single-dose cosmetic emulsion.

In other words, the invention consists in a device intended for the preparation, immediately before application on the skin, skin appendages or mucous membranes, of a single-use single-dose cosmetic composition, i.e. of a cosmetic composition immediately applicable and in its entirety on the skin, the skin appendages or the mucous membranes.

The cosmetic emulsion is obtained immediately after the mixture of the invention device with water. No additional ingredients are necessary.

The term “single-dose” means a sufficient amount of cosmetic emulsion to be applied in its entirety and at once on the skin, the skin appendages or the mucous membranes.

Similarly, the term “consumable” means that all the constituent elements of the device help form the final cosmetic emulsion.

The term “anhydrous” means a composition free or substantially free of water.

The consumable anhydrous device is in practice in the form of a closed shell, constituting an ingredient of the cosmetic emulsion and containing a fatty phase corresponding to the remaining ingredients of said cosmetic emulsion, with the exception of water.

Therefore the invention involves a consumable anhydrous device in the form of a closed shell containing a fatty phase, the shell and the fatty phase constituting the necessary ingredients and in an amount adapted for the extemporaneous preparation, solely by adding water, of a single-use cosmetic emulsion.

According to an essential characteristic of the invention, all constituents of the consumable anhydrous device are part of the final cosmetic formulation, the shell included.

According to the invention, when the device is mixed with an appropriate amount of water, it forms an oil-in-water emulsion wherein the shell is completely consumed.

Depending on embodiments of the invention, the shell can be a capsule or a sealed sachet.

The ingredients contained in the fatty phase are, in practice, selected from the cosmetic active ingredients, synthetic oils, vegetable oils, emollients, anti-UV filters, texturizing agents, thickeners, gelling agents, pigments or dyes, aromas or fragrances, surfactants, but are not limited to this list. The person skilled in the art will know how to select the ingredients necessary to the formation of an emulsion after contact with water.

According to an essential characteristic, the entire consumable device, the shell and its contents, are required to obtain the finished cosmetic product, and contribute both to the technical quality and sensory properties of the finished cosmetic product. The shell serves, among other things, as a gelling agent and/or texturizing agent; it is entirely consumed and allows the method according to the invention not to generate waste.

To this end, the shell can be made from one or more components from among gelatin, hydroxy-propyl methyl cellulose, natural or synthetic polymers, pullulan or gellan gum. The capsules marketed by CAPSUGEL-France under the trade names of “Plantacaps™” made out of pullulan, “Vcaps®” made out of hydroxy-propyl methyl cellulose, “Licaps®” made out of gelatin or “DRcaps™” made out of a mixture of gellan gum (5%) and hydroxy-propyl methyl cellulose (qsp 100%) may especially be used for this purpose.

The invention also provides a kit for the implementation of the preparation method according to the invention, which is characterized in that it comprises:
at least one consumable anhydrous device; and.
a container adapted to get water and the device in contact, and to produce the mixture allowing to form the emulsion.

In a particular embodiment, the container contained in this kit may show at least a visual or graduation mark indicating to the user the amount of water to be poured into the container.

According to another embodiment, this container can include a sealing means which, when it is removed or is in open position, allows the introduction of water and the consumable anhydrous device, and which, when it is positioned or is in closed position, seals the container and allows for the production of the mixture by shaking the container.

In yet another embodiment, the container may comprise at least a means of improving the mixture which facilitates the formation of the emulsion.

The invention also relates to a method for preparing an extemporaneous single-dose single-use cosmetic emulsion implementing the device of the invention. This method is intended to be implemented by the consumer of the cosmetic emulsion just prior to applying said emulsion to the skin, skin appendages or mucous membranes.

The method is characterized in that it includes the following steps:
providing a consumable anhydrous device comprising a fatty phase,
providing water, in a predetermined amount, corresponding to the amount necessary for the preparation of the single-use cosmetic emulsion;
putting the water in contact with the device and mixing the whole together to form a single-use single-dose cosmetic emulsion applicable directly, immediately, and in its entirety on the skin, the skin appendages or the mucous membranes.

The method according to the invention allows to simply make any cosmetic product in “oil in water” type emulsion form, be it a cream, milk, gel, or any other conceivable form of emulsion to make a cosmetic product intended to be applied on any part of the human body, in particular the skin (of the body or of the face), the hair or nails or mucous membranes.

According to other embodiments of the invention, one can heat the water before putting it in contact with the device, and/or heat the mixture during the mixing step, to facilitate the formation of the emulsion, and/or cool the emulsion obtained before applying it on the skin. The water temperature may be in the order of 60 to 70° C.

The method according to the invention allows therefore, for instance, to produce a moisturizing product (facial day or night cream, body lotion, hand cream, lip balm . . . ), treatment product (anti-wrinkle, anti-acne, concealer . . . ), a hair product (anti-dandruff, anti-fall, strengthening, dye or bleach, conditioner . . . ), a sunscreen or self-tanning product, a body or face scrub, a skin or hair mask, a makeup or makeup removal product, a shaving or waxing product, a perfuming product, a bathroom product such as shampoo, conditioner, shower gel, foaming or other bath product, a massage gel, or any other cosmetic products.

The method according to the invention is intended to be implemented directly by the user of the cosmetic product at home. It is therefore particularly simple, so that it can be done by anyone without special training or habit or practice, and without any specific equipment.

It may, nevertheless, without departing from the scope of this invention, be performed by a professional in a beauty salon, a hair salon, manicure salon, make-up or esthetics salon, a wellness, relaxation or massage center, a cosmetics store or any other similar structure wherein a cosmetic product could be prepared in order to be applied to the body of a consumer.

The ingredients in the fatty phase of the anhydrous device may be in a solid or liquid or pasty form. In all cases, the anhydrous device is free or substantially free of water, the water necessary for the preparation of the emulsion being added later.

From the consumable anhydrous device, the method according to the invention is very easy to implement. Thus simply provide an amount of water adapted for the preparation of a dose of the desired cosmetic product. This water amount is predetermined in advance depending on the nature and bulk of the anhydrous composition, including the shell. It further depends on the nature of the cosmetic product one wishes to obtain, for instance, obtaining a dose of lotion which would obviously require more water than obtaining a dose of cream.

When in contact with water, the shell of the anhydrous composition swells, disintegrates and/or dissolves. The other ingredients are then released and are found in the water. By stirring the anhydrous device in the water, an oil-in-water type emulsion is formed which constitutes the final cosmetic product. The single-dose of cosmetic product obtained is then ready to be used.

The invention and advantages deriving therefrom are highlighted by the exemplary embodiments hereafter.

5 mixtures of distinct cosmetic ingredients have been prepared. Each mixture has been filled into 4 different capsules.

The capsules used are as follows and have been supplied by the company CAPSUGEL:

Plantcaps® (100% pullulan)
Vcaps® (100% hypromellose)
Licaps® (gelatin)
DRcaps® (5% gellan gum+hypromellose qsp 100%)

### EXAMPLE 1

Four identical formulations containing the composition detailed in the table below are prepared.

| | | Composition |
|---|---|---|
| Ingredients | Function | (in percentage by weight) |
| Emulium Delta | emulsifier | 37.5 |
| Labrafac CC | oil | 37.5 |
| DPPG | texturing agent | 25 |

The compositions thus obtained are filled into each of the 4 aforementioned capsules. Each anhydrous composition thus obtained is mixed with 6 g of 65° C. hot water.

With Licaps® capsule, a single-dose of cream is obtained.

With Vcaps® capsule, a single-dose of lotion is obtained.

With Plantcaps® capsule, a single-dose of cream is obtained.

With DRcaps® capsule, a single-dose of cream is obtained.

### EXAMPLE 2

Four identical formulations containing the composition detailed in the table below are prepared.

| | | Composition |
|---|---|---|
| Ingredients | Function | (in percentage by weight) |
| Emulium Delta | emulsifier | 23 |
| Labrafac CC | oil | 22 |
| Jojoba oil | oil | 11 |
| DPPG | texturing agent | 15 |
| Xiameter PMX-0345 | texturing agent | 27 |
| Gatuline In-Tense | active ingredient | 2 |

The compositions thus obtained are filled into each of the 4 aforementioned capsules. Each anhydrous composition thus obtained is mixed with 5 g of 65° C. hot water.

With Licaps® capsule, a single-dose of cream is obtained.

With Vcaps® capsule, a single-dose of lotion is obtained.

With Plantcaps® capsule, a single-dose of cream is obtained.

With DRcaps® capsule, a single-dose of cream is obtained.

### EXAMPLE 3

Four identical formulations containing the composition detailed in the table below are prepared.

| | | Composition |
|---|---|---|
| Ingredients | Function | (in percentage by weight) |
| Emulium 22 | emulsifier | 25 |
| MOD | emollient | 29 |
| Cetiol OE | emollient | 17 |
| Geleol | texturing agent | 12 |
| Dimethicone Fluid 100 | texturing agent | 17 |

The compositions thus obtained are put into each of the 4 aforementioned capsules. Each anhydrous composition thus obtained is mixed with 5 g of 65° C. hot water.

With Licaps® capsule, a single-dose of cream is obtained.

With Vcaps® capsule, a single-dose of lotion is obtained.

With Plantcaps® capsule, a single-dose of cream is obtained.

With DRcaps® capsule, a single-dose of cream is obtained.

### EXAMPLE 4

Four identical formulations containing the composition detailed in the table below are prepared.

| | | Composition |
|---|---|---|
| Ingredients | Function | (in percentage by weight) |
| Emulium kappa | emulsifier | 18 |
| Cetiol OE | emollient | 23 |
| Eutanol G | emollient | 27 |
| Isostearyl isostearate | emollient | 18 |
| Lipocire A | texturing agent | 14 |

The compositions thus obtained are filled into each of the 4 aforementioned capsules. Each anhydrous composition thus obtained is mixed with 5 g of 65° C. hot water.

With Licaps® capsule, a single-dose of cream is obtained.

With Vcaps® capsule, a single-dose of lotion is obtained.

With Plantcaps® capsule, a single-dose of cream is obtained.

With DRcaps® capsule, a single-dose of lotion-cream is obtained.

### EXAMPLE 5

Four identical formulations containing the composition detailed in the table below are prepared.

| | | Composition |
|---|---|---|
| Ingredients | Function | (in percentage by weight) |
| Emulium kappa | emulsifier | 22 |
| Jojoba oil | oil | 27 |
| Cetiol OE | emollient | 30 |
| Geleol | texturing agent | 9 |
| Lipocire A | texturing agent | 6 |
| Lavender essential oil | active ingredient | 6 |

The compositions thus obtained are filled into each of the 4 aforementioned capsules. Each anhydrous composition thus obtained is mixed with 5 g of 65° C. hot water.

With Licaps® capsule, a single-dose of cream is obtained.

With Vcaps® capsule, a single-dose of lotion is obtained.

With Plantcaps® capsule, a single-dose of cream is obtained.

With DRcaps® capsule, a single-dose of cream is obtained.

Obviously, this invention is not limited to the preferred embodiments previously described, since a person skilled in the art can make numerous modifications thereto and imagine other alternatives without departing from the scope or framework of the invention defined by the claims.

1. An anhydrous device being in the form of a closed shell containing a fatty phase, the shell and the fatty phase constituting the necessary ingredients and in an amount adequate for the extemporaneous preparation, solely by adding water, of a single-use cosmetic emulsion.

2. The device according to claim 1, wherein the device is a capsule, a casing, or a sealed sachet.

3. The device according to claim 1, wherein the fatty phase comprises one or more ingredients selected from cosmetic active ingredients, synthetic oils, vegetable oils, emollients, anti-UV filters, texturing agents, thickening agents, gelling agents, pigments or dyes, aromas or fragrances, and surfactants.

4. The device according to claim 1, wherein the shell is made from one or more components selected from gelatin, hydroxy-propyl methyl cellulose, natural or synthetic polymers, pullulan and gellan gum.

5. The device according to claim 1, wherein the shell serves as gelling agent and/or texturing agent.

6. The device according to claim 1, wherein ingredients contained in the fatty phase are in a solid, liquid or pasty form.

7. A method for extemporaneous preparation of a single-use cosmetic emulsion comprising implementing the device according to claim 1.

8. The method according to claim 7, said method comprising the following steps:
providing the consumable anhydrous device comprising a fatty phase,
providing water, in a predetermined amount, corresponding to the amount necessary for the preparation of the single-use cosmetic emulsion; and
putting water in contact with the device and mixing the whole to form a single-use cosmetic emulsion applicable directly, immediately and in its entirety on skin, skin appendages or mucous membranes.

9. The method of claim 8, wherein the water is heated before putting it in contact with the consumable anhydrous device and/or heating the mixture during the mixing step to facilitate the formation of the emulsion, and/or cooling or allowing the emulsion obtained to cool down before applying it on the skin.

10. The method according to claim 7, wherein the cosmetic emulsion obtained is either a cream or a lotion.

11. The device according to claim 2, wherein the fatty phase comprises one or more ingredients selected from cosmetic active ingredients, synthetic oils, vegetable oils, emollients, anti-UV filters, texturing agents, thickening agents, gelling agents, pigments or dyes, aromas or fragrances, and surfactants.

12. The device according to claim 11, wherein the shell is made from one or more components selected from gelatin, hydroxy-propyl methyl cellulose, natural or synthetic polymers, pullulan and gellan gum.

13. The device according to claim 2, wherein the shell is made from one or more components selected from gelatin, hydroxy-propyl methyl cellulose, natural or synthetic polymers, pullulan and gellan gum

14. The device according to claim 3, wherein the shell is made from one or more components selected from gelatin, hydroxy-propyl methyl cellulose, natural or synthetic polymers, pullulan and gellan gum

15. The device according to claim 14, wherein the shell serves as gelling agent and/or texturing agent.

16. The device according to claim 2, wherein the shell serves as gelling agent and/or texturing agent.

17. The device according to claim 3, wherein the shell serves as gelling agent and/or texturing agent.

18. The device according to claim 2, wherein ingredients contained in the fatty phase are in a solid, liquid or pasty form.

19. The device according to claim 15, wherein ingredients contained in the fatty phase are in a solid, liquid or pasty form.

20. The method according to claim 7, said method comprising the following steps:
providing the consumable anhydrous device comprising a fatty phase,
providing water, in a predetermined amount, corresponding to the amount necessary for the preparation of the single-use cosmetic emulsion; and
putting water in contact with the device and mixing the whole to form a single-use cosmetic emulsion applicable directly, immediately and in its entirety on skin, skin appendages or mucous membranes,
wherein the cosmetic emulsion obtained is either a cream or a lotion.
